SEQUENCE LISTING (3 sequences)

SEQ ID NO: 1 - protein, 77 aa, Homo sapiens
AVLPRSAKELRCQCIKTYSKPFHPKFIKELRVIESGPHCANTEIIVKLSDGRELCLDPKENWVQRVVEKFLKRAENS

SEQ ID NO: 2 - protein, 10 aa, Artificial Sequence
  Peptide containing a Factor Xa cleavage site and a Protein Kinase A site.
GIEGRRRASV

SEQ ID NO: 3 - protein, 8 aa, Artificial Sequence
  His(6) tag peptide.
LEHHHHHH

(19) United States
(12) Patent Application Publication
(10) Pub. No.: US 2005/0233473 A1
(43) Pub. Date: Oct. 20, 2005

(54) Methods and reagents for surface functionalization

(75) Inventors: Ronald Cicero, Eugene, OR (US); Jonathan Forman, San Carlos, CA (US); Peter Kernen, Foster City, CA (US); Hongbo Lu, Fremont, CA (US); David Quincy, Fremont, CA (US); Stefan Schweizer, Hayward, CA (US); Peter Wagner, Emerald Hills, CA (US)

Correspondence Address: KLARQUIST SPARKMAN, LLP, 121 SW SALMON STREET, SUITE 1600, PORTLAND, OR, 97204, US

(73) Assignee: Zyomyx, Inc.

(21) Appl. No.: 10/980,597

(22) Filed: Nov. 2, 2004

Related U.S. Application Data
(63) Continuation-in-part of application No. PCT/US03/25927, filed on Aug. 18, 2003.
(60) Provisional application No. 60/403,971, filed on Aug. 16, 2002.
(60) Provisional application No. 60/518,398, filed on Nov. 4, 2003.

(51) Int. Cl. G01N 33/543

(52) U.S. Cl. 436/518

(57) ABSTRACT

Reagents and a method for making arrays of affinity agents are disclosed. Methods of using the arrays of affinity agents also are disclosed. The arrays are particularly useful for high throughput drug screening and clinical diagnostics applications.

### CROSS REFERENCE TO RELATED APPLICATIONS

This application is a continuation-in-part of International Application No. PCT/US03/025927, filed Aug. 18, 2003, which was published in English under PCT Article 21(2), which claims the benefit of U.S. Provisional Application No. 60/403,971, entitled, “Biochip Surface Configurations and Methods and Compositions for Making the Same,” filed Aug. 16, 2002, in the name of David Quincy. This application also claims the benefit of U.S. Provisional Application No. 60/518,398, entitled, “Methods and Reagents for Surface Functionalization,” filed Nov. 4, 2003. Each of these prior applications is incorporated herein by reference in its entirety.

### FIELD

This disclosure concerns a class of reagents and a method for surface functionalization for creating arrays of bioactive compounds on a substrate.

### BACKGROUND

Comprehensive understanding of disease states requires the identification and characterization of all proteins involved in a particular disease pathway. This study of proteins and their interactions is termed “proteomics.” Due to advances in molecular biology, proteins of interest can be produced more rapidly than current techniques can characterize the proteins. For example, conventional methods for protein identification in proteomics applications rely upon two-dimensional, polyacrylamide gel electrophoresis (2D-PAGE) technology to isolate proteins, followed by subsequent identification by mass spectrometry. Typically, 2D-PAGE can separate as many as 5000 different proteins. However, identification of each protein is a tedious, labor-intensive process that requires cutting each of the individual separated proteins out of the polyacrylamide gel. Moreover, the method has a relatively low sensitivity. For example, when using silver staining, the detection limit is about 1 nanogram of protein.

Additional drawbacks of 2D-PAGE include a lack of reproducibility, low throughput, low resolution and protein-dependent sensitivity. For example, 2D-PAGE generally does not resolve all proteins present in a mixture and systems are limited to processing a handful of gels over a two-day period. In addition, high-molecular-weight, low-molecular-weight and membrane-bound proteins are underrepresented.

Attempts have been made to use protein arrays for the high throughput characterization of proteins. For example, U.S. Pat. No. 6,406,921 to Wagner et al. discloses a method of making protein-coated substrates, and U.S. Pat. No. 5,620,850 to Bamdad et al. discloses a method for making a surface including a plurality of chelating agents, which can be used to bind metal ions. The bound metal ions are then reportedly used to capture a biological molecule that also includes a chelating agent.

A number of hurdles must be overcome to provide protein arrays of high quality which produce accurate and reproducible screening results. Typically, proteins must remain hydrated, be kept at ambient temperatures, and are very sensitive to the physical and chemical properties of the support materials. Thus, maintaining protein activity at the liquid-solid interface requires new strategies for assembling arrays that address the sensitivity of the proteins to the environment.

### SUMMARY

Disclosed herein are reagents and a method for using such reagents to prepare substrates derivatized with affinity agents, such as small molecules, peptides, proteins, nucleic acids and other bioactive molecules. As disclosed herein, densely packed affinity agent arrays exhibiting minimal, non-specific binding can be prepared.

In one aspect of the method, a substrate is functionalized with a reagent having at least two reactive functional groups. The first reactive functional group serves to couple the reagent to the substrate and the second reactive functional group is an affinity agent or provides a site for operatively associating an affinity agent.

In a second aspect of the method, non-specific protein adhesion to the arrays is diminished by the incorporation of a protein resistant component, which diminishes such non-specific protein adhesion. Thus, in one aspect, the disclosure provides reagents and techniques for assembling arrays of affinity agents that exhibit reduced non-specific binding.

### BRIEF DESCRIPTION OF THE DRAWINGS

FIG. 1 illustrates one embodiment of the disclosed method for functionalizing a surface.

FIG. 2A is a schematic representing one embodiment of the disclosed array.

FIG. 2B provides examples of monolayer components used to prepare such arrays.

FIG. 3A illustrates a method for using an asymmetric disulfide reagent to prepare a monolayer.

FIG. 3B depicts examples of monolayer components prepared via Staudinger ligation.

FIG. 4 illustrates one example of a process for preparing a novel array.

FIG. 5 is a schematic of a fluorescence detection unit which may be used to monitor interaction of the proteins of the array with an analyte.

FIG. 6 is a schematic of an ellipsometric detection unit which may be used to monitor interactions between analytes and the affinity tags of the array.

FIG. 7 illustrates the synthesis of an asymmetric disulfide having N-hydroxysuccinimide and polyethylene glycol groups.

### DETAILED DESCRIPTION

The following explanations of terms and methods are provided to better describe the present compounds, compositions and methods and to guide those of ordinary skill in the art in the practice of the present disclosure. The terminology used in the disclosure is for the purpose of describing particular embodiments and examples only and is not intended to be limiting.

### A. Definitions

The term “substrate” as used herein refers to a bulk, underlying material used in the arrays and devices disclosed herein.

An “array” refers to a two-dimensional distribution or pattern.

The terms “polypeptide” and “protein” are used interchangeably to refer to an amino acid polymer.

The term “antibody” means an immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. These proteins may be derived from natural sources, or partly or wholly synthetically produced. An antibody may be monoclonal or polyclonal. In an exemplary embodiment, the antibody is a glycosylated antibody. The antibody may be a member of any immunoglobulin class, including any of the human classes: IgG, IgM, IgA, IgD, and IgE. In an exemplary embodiment, the antibody is of the IgG class.

The term “antibody fragment” refers to any derivative of an antibody which is less than full-length. In an exemplary embodiment, the antibody fragment retains at least a significant portion of the full-length antibody's specific binding ability. Examples of antibody fragments include, but are not limited to, Fab, Fab′, F(ab′)₂, scFv, Fv, dsFv diabody, and Fd fragments. The antibody fragment may be produced by any means. For instance, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

Single-chain Fvs (scFvs) are recombinant antibody fragments consisting of only the variable light chain (V_{L}) and variable heavy chain (V_{H}) covalently connected to one another by a polypeptide linker. Either V_{L} or V_{H} may be the NH₂-terminal domain. The polypeptide linker may be of variable length and composition so long as the two variable domains are bridged without serious steric interference. Typically, the linkers are comprised primarily of stretches of glycine and serine residues with some glutamic acid or lysine residues interspersed for solubility.

A Fv fragment is an antibody fragment which consists of one V_{H} and one V_{L} domain held together by noncovalent interactions. The term “dsFv” is used herein to refer to an Fv with an engineered intermolecular disulfide bond to stabilize the V_{H}-V_{L} pair.

A F(ab′)₂ fragment is an antibody fragment essentially equivalent to that obtained from immunoglobulins (typically IgG) by digestion with an enzyme pepsin at pH 4.0-4.5. The fragment may be recombinantly produced.

A Fab′ fragment is an antibody fragment essentially equivalent to that obtained by reduction of the disulfide bridge or bridges joining the two heavy chain pieces in the F(ab′)₂ fragment. The Fab′ fragment may be recombinantly produced.

A Fab fragment is an antibody fragment essentially equivalent to that obtained by digestion of immunoglobulins (typically IgG) with the enzyme pepsin. The Fab fragment may be recombinantly produced.

“Amino acid” refers to both naturally occurring and “unnatural” amino acids. Residues of amino acids also are encompassed by the term amino acid.

“Organic thinfilm” refers to a thin layer of organic molecules formed directly on a substrate or on a coating on the substrate. Typically, the organic thinfilms disclosed herein are from about 0.5 nm to about 50 nm thick, and more typically from about 0.5 nm to about 10 nm thick. The organic thinfilm can be assembled prior to deposition on the substrate; however, typically the organic thinfilm is assembled as the component molecules are attached to the substrate. The organic thinfilm optionally includes associated inorganic ions and chelated metals bound to the thinfilm. The organic thinfilm can be homogeneous or heterogeneous and can be composed of one or more monolayers. An example of a thinfilm composed of plural monolayers is a lipid bilayer. However, typically the organic thinfilm is a monolayer. Optionally, the organic thinfilm can include more than one type of organic thinfilm.

Typically, the organic thinfilms disclosed herein include an affinity agent or a functional group suitable for covalently or noncovalently associating an affinity agent to the thinfilm. The organic thinfilm also optionally can bear functional groups that reduce the association of molecules with the thinfilm. Typically, such functional groups are hydrophilic groups, such as, for example, polyalkylene oxides, including polyethylene glycol (PEG) and polypropylene glycol (PPG), which generally are not bound tightly by proteins. PEG and PPG include oligomers of ethylene glycol and propylene glycol, respectively. As such, PEG and PPG as used herein refer to polymers having as few as two glycol subunits. In working embodiments, PEG was used as a protein-resistant component of organic thinfilms to reduce the nonspecific binding of proteins to the organic thinfilm. In several working examples, PEG components that were used were not monodisperse, and therefore the specific numbers of ethylene glycol units referred to can also refer to an average number where a polydisperse mixture of PEG units are used. Other functional groups of the organic thinfilm serve to tether the thinfilm to the surface of the substrate or a coating on the substrate.

The term “monolayer” refers to a layer having a single-molecule thickness, which can be an organic thinfilm or portion thereof. A monolayer can be ordered or disordered. Typically the monolayer is ordered and densely packed. The monolayer can be homogeneous or heterogeneous, however one face of the monolayer will include functional groups that can be chemisorbed or physisorbed onto the surface of the substrate or a coating on the substrate.

A first monolayer also can be converted into a second monolayer. For example, the component molecules in a first monolayer having a first end chemisorbed or physisorbed to a substrate can be functionalized by covalently bonding one or more second molecules to plural second ends of the first monolayer component molecules, thereby converting the first monolayer into a new, second monolayer.

### B. Arrays of Affinity Agents

The present disclosure is directed to arrays of affinity agents and methods for making and using such arrays. Typically the arrays form a two-dimensional display of an affinity agent, which can be used to characterize the interaction of the affinity agent with a soluble molecule of interest.

Thus, in one embodiment, the disclosed functionalized substrates are further functionalized with an affinity agent. The affinity agent can be any agent that can be bound to the functionalized substrate and that interacts covalently or noncovalently with a molecule of interest. Typically the affinity agent is a small molecule, oligonucleotide, peptide or protein that binds to or interacts with a soluble molecule of interest. Similarly, the soluble molecule of interest or analyte can be a small molecule, oligonucleotide, peptide or protein. Interactions between the affinity agent and the analyte can be detected by any suitable method, and working embodiments used methods such as optical detection methods including ultraviolet and visible absorption, chemoluminescence, and fluorescence (including lifetime, polarization, fluorescence correlation spectroscopy (FCS), and fluorescence-resonance energy transfer (FRET)).

In one embodiment, an array of affinity agents includes a substrate, an organic thinfilm formed on at least a portion of the surface of the substrate, and including plural copies of at least one affinity agent operatively associated with, e.g., covalently or noncovalently associated with, the underlying organic thinfilm.

Generally, when plural types of affinity agents are used in the same array, the different types of affinity agents are grouped in “patches,” so that affinity agents localized in one patch of the array differ from affinity agents localized to another patch. In one embodiment, the present invention provides an array of glycoproteins containing a substrate, at least one organic thinfilm on some or all of the substrate surface, and a plurality of patches arranged in discrete, known regions on portions of the substrate surface covered by organic thinfilm, wherein each of said patches comprises a protein immobilized on the underlying organic thinfilm. The array optionally contains an interlayer between the substrate and coating.

In most cases, the array will comprise at least about ten patches. In an exemplary embodiment, the array comprises at least about 50 patches. In another exemplary embodiment the array comprises at least about 100 patches. In alternative exemplary embodiments, the array of affinity agents can comprise more than 10³, 10⁴ or 10⁵ patches.

In an exemplary embodiment, the surface area of the substrate covered by each of the patches is no more than about 0.25 mm². In another exemplary embodiment, the area of the substrate surface covered by each of the patches is between about 1 μm² and about 10,000 μm². In another exemplary embodiment, each patch covers an area of the substrate surface from about 100 μm² to about 2,500 μm². In an alternative embodiment, a patch on the array may cover an area of the substrate surface as small as about 2,500 nm², although patches of such small size are generally not necessary for the array to be useful.

The patches of the array may be of any geometric shape. For instance, the patches may be rectangular or circular. The patches of the array may also be irregularly shaped.

The distance separating the patches of the array can vary. For example, the patches of the array are separated from neighboring patches by about 1 μm to about 500 μm. Typically, the distance separating the patches is roughly proportional to the diameter or side length of the patches on the array if the patches have dimensions greater than about 10 μm. If the patch size is smaller, then the distance separating the patches typically will be larger than the dimensions of the patch.

In an exemplary embodiment of the array, the patches of the array are all contained within an area of about 1 cm² or less on the surface of the substrate. In one exemplary embodiment of the array, therefore, the array comprises 100 or more patches within a total area of about 1 cm² or less on the surface of the substrate. Alternatively, an exemplary array comprises 10³ or more patches within a total area of about 1 cm² or less. An exemplary array may even optionally comprise 10⁴ or 10⁵ or more patches within an area of about 1 cm² or less on the surface of the substrate. In other embodiments of the invention, all of the patches of the array are contained within an area of about 1 cm² or less on the surface of the substrate.

In one embodiment, only one type of affinity agent is immobilized on each patch of the array. In an exemplary embodiment of the array, the affinity agent immobilized on one patch differs from the affinity agent immobilized on a second patch of the same array. In such an embodiment, a plurality of different affinity agents can be present on separate patches of the array. In another aspect, a single patch comprises two or more affinity agents that bind to the same analyte. Such affinity agents typically bind to different epitopes of the analyte. One class of affinity agents that can be used in this aspect is polyclonal antibodies.

Typically the array comprises at least about ten different affinity agents. In an exemplary embodiment, the array comprises at least about 50 different affinity agents. In another exemplary embodiment, the array comprises at least about 100 different affinity agents. Alternative exemplary arrays comprise more than about 10³ different affinity agents or more than about 10⁴ different affinity agents. The array may even optionally comprise more than about 10⁵ different affinity agents.

In one embodiment of the array, each of the patches of the array contains a different affinity agent. For instance, an array comprising about 100 patches could comprise about 100 different affinity agents. Likewise, an array of about 10,000 patches could comprise about 10,000 different affinity agents. In an alternative embodiment, however, each different affinity agent is immobilized on more than one separate patch on the array. For instance, each different affinity agent can optionally be present on two to six different patches. Therefore an array can comprise about three-thousand affinity agent patches, but only comprise about one thousand different affinity agents, since each different agent is present on three different patches.

In another embodiment, although the affinity agent of one patch is different from that of another, the affinity agents are related. In an exemplary embodiment, the two different affinity agents are members of the same protein family. The different proteins on the array can be either functionally related or thought to be functionally related. In another embodiment of the invention array, however, the function of the immobilized proteins may be unknown. In this case, the different glycoproteins on the different patches of the array typically share a similarity in structure or sequence or are thought to sharing a similarity in structure or sequence. Alternatively, the immobilized proteins can be fragments of different members of a protein family.

Any affinity agent that can be operatively associated with an organic thinfilm on the substrate can be employed in the disclosed arrays. Classes of different affinity agents include, without limitation, small molecules, peptides, proteins and nucleic acids, including DNA and RNA. Optionally, an array can include different affinity agents from different classes.

When proteins are selected as affinity agents, the proteins can be members of a protein family, such as a receptor family, examples of which include growth factor receptors, catecholamine receptors, amino acid derivative receptors, cytokine receptors, and lectins; a ligand family, examples of which include cytokines and serpins; an enzyme family, examples of which include proteases, kinases, phosphatases, ras-like GTPases, and hydrolases; and transcription factors, examples of which include steroid hormone receptors, heat-shock transcription factors, zinc-finger proteins, leucine-zipper proteins and homeodomain proteins. In one embodiment, the different immobilized proteins are all HIV proteases or hepatitis C virus (HCV) proteases. In another embodiment the associated proteins on the array are all hormone receptors, neurotransmitter receptors, extracellular matrix receptors, antibodies, DNA-binding proteins, intracellular signal transduction modulators and effectors, apoptosis-related factors, DNA synthesis factors, DNA repair factors, DNA recombination factors, or cell-surface antigens.

Antibodies and antibody fragments are particularly useful affinity agents for use with the disclosed arrays. The antibodies optionally can be polyclonal or monoclonal antibodies. The production and isolation of antibodies that bind to specific targets, including protein targets, using standard hybridoma technology is known to those of ordinary skill in the art. Moreover, numerous antibodies are available commercially. Alternatively, antibodies or antibody fragments can be expressed in bacteriophage. Such antibody phage display technologies, including methods for bacteriophage selection, are well known to those of ordinary skill in the art.

### C. Reagents and Techniques for Preparing Arrays of Affinity Agents

In one aspect, the disclosure provides reagents and techniques for assembling arrays of affinity agents. According to one embodiment of the method a first heterobifunctional reagent is covalently or non-covalently associated with a substrate, thereby forming a monolayer. A second heterobifunctional reagent is coupled to the monolayer to provide an array of reactive functional groups and a third reagent including a protein resistant component is coupled to the monolayer, such that the three reagents form an organic thinfilm including reactive functional groups. The reactive functional groups are selected such that an affinity agent can be coupled to the organic thinfilm, thereby forming an affinity agent array having affinity agents operatively coupled to the organic thinfilm. Coupling involves covalently or non-covalently associating the affinity agent. Non-covalent associate may exploit, without limitation, one or more of Coulombic interactions, hydrogen bonds, Van der Waals interactions and hydrophobic interactions.

In one embodiment, an affinity agent is coupled to an organic thinfilm by a chemoselective ligation reaction. Chemoselective ligation reactions generally refer to reactions between functional groups that have orthogonal reactivity to other functional groups present, particularly those functional groups found in many biomolecules. Thus, chemoselective ligation reactions are particularly useful when the affinity agent is a biomolecule. Typically, chemoselective ligation reactions used with biomolecules employ one or more non-native functional groups to ensure that the reaction is orthogonal to native functional groups. One example of a chemoselective ligation reaction is a Staudinger ligation. Other examples include reactions of ketones and aldehydes, such as the condensation of a hydrazide or aminooxy compound with a ketone or aldehyde to yield the corresponding hydrazone or oxime. Another example is the reaction of a thiocarboxylate with an α-halo carbonyl compound to give a thioester. Versions of these reactions also can be used to attach compounds other than biomolecules with the organic thinfilm.

Exemplary methods disclosed herein employ a Staudinger ligation reaction to associate a reagent or affinity agent with an organic thinfilm. Staudinger ligation functions as an amide bond forming reaction and typically involves two reactive components, the first typically having the formula Y-Z-PR²R³ where Z is an aryl group substituted with R¹, wherein R¹ is preferably in the ortho position on the aryl ring relative to the PR² R³; and wherein R¹ is an electrophilic group to trap (e.g., stabilize) an aza-ylide group, including, but not necessarily limited to, a carboxylic acid, an ester (e.g., alkyl ester (e.g., lower alkyl ester, benzyl ester), aryl ester, substituted aryl ester), aldehyde, amide, e.g., alkyl amide (e.g., lower alkyl amide), aryl amide, alkyl halide (e.g., lower alkyl halide), thioester, sulfonyl ester, alkyl ketone (e.g., lower alkyl ketone), aryl ketone, substituted aryl ketone, halosulfonyl, nitrile, nitro and the like; R² and R³ are generally aryl groups, including substituted aryl groups, or cycloalkyl groups (e.g., cyclohexyl groups) where R² and R³ may be the same or different, preferably the same; and Y is H, a protein resistant group, a reactive group that facilitates covalent attachment of an affinity agent or a molecule of interest, wherein Y can be at any position on the aryl group (e.g., para, meta, ortho); where exemplary reactive groups include, but are not necessarily limited to, carboxyl, amine, (e.g., alkyl amine (e.g., lower alkyl amine), aryl amine), ester (e.g., alkyl ester (e.g., lower alkyl ester, benzyl ester), aryl ester, substituted aryl ester), thioester, sulfonyl halide, alcohol, thiol, succinimidyl ester, isothiocyanate, iodoacetamide, maleimide, hydrazine, and the like. Exemplary affinity agents further include dyes (e.g., fluorescein or modified fluorescein, and the like), antibodies, toxins (including cytotoxins), linkers, peptides, and the like. An exemplary and preferred engineered phosphine reactant is 2-diphenylphosphanyl-benzoic acid methyl ester.

The second reagent comprises an azide. Molecules comprising an azide and suitable for use in the present invention, as well as methods for producing azide-comprising molecules suitable for use in the present invention, are well known to those having ordinary skill in the art.

In a working embodiment a monolayer comprising a 2-diphenylphosphanyl-benzoic acid methyl ester derivative was prepared on a gold substrate. According to this embodiment, a first reagent, 11-thio-undecanionic-N-hydroxysuccinimide ester was linked to the substrate via the sulfhydryl group. The resulting monolayer was derivatized with compound 25, thereby forming a second monolayer from the first, where the second monolayer is suitable for coupling of an affinity agent containing an azide moiety. A fluorescently labeled, azidoalanine-containing peptide was then coupled to the second monolayer via Staudinger ligation.

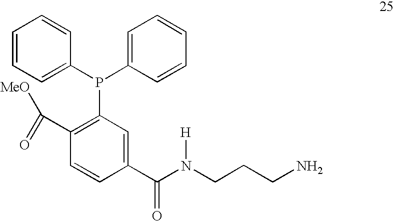

In another aspect of the method, a native functional group of a biomolecule is used to attach it to an organic thinfilm. For example, proteins that have one or more cysteine residues can be selectively alkylated with reagents, such as α-halo carbonyl compounds. When an α-halo carbonyl compound is associated with a thinfilm, this can be a useful reaction for associating certain proteins to the thinfilm.

In a parallel method, a natural or non-natural amino acid can be incorporated into a peptide or protein to provide a functional group for associating the protein with an organic thinfilm. For example, as discussed above, in a working embodiment an azide-containing amino acid was incorporated into a peptide, which is then attached to an organic thinfilm via a Staudinger ligation protocol.

In one embodiment, the substrate is functionalized with a chelator, such as an N-nitrilotriacetic acid (NTA) derivative or an imidodiacetic acid (IDA) derivative, which chelate metals, such as nickel, cobalt, iron and copper. Using such derivatives, a histidine-tagged protein can be noncovalently bound to the substrate via, for example, mutual nickel chelation by both the substrate-associated chelator, such as an NTA or IDA derivative, and the histidine tag. Proteins having histidine tags are known to those of ordinary skill in the art. See, Hochuli, et al., Biotechnology, 1988, 6, 1321. In another aspect of this method for attaching an affinity agent, the initial, non-covalent association can be followed by covalent bond formation between the affinity agent and the substrate associated organic thinfilm. This can be accomplished by using, for example a photoactivatable group, such as an aryl azide, particularly haloaryl azides, for example a pentafluorophenyl aryl azide, benzophenones, diazocompounds, particularly diazopyruvates. Additional suitable photoactivatable groups are taught by Hermanson, G. T. Bioconjugate Techniques, Academic Press: San Diego, 1996, which is incorporated herein by reference.

In a second example, the first affinity agent is a multivalent protein, and is associated with a substrate-associated organic thinfilm. The first affinity agent can then serve as the site of attachment for a second affinity agent having a portion for binding to the first affinity agent and a portion for interacting with a molecule of interest. In a specific example, the first affinity agent is streptavidin, which is bound to the substrate via a biotin derivative displayed on the substrate-associated organic thinfilm, and the second affinity agent is a biotin-conjugated molecule. Because streptavidin has plural biotin binding sites, the biotin conjugated molecule is then associated with the organic thinfilm via the streptavidin.

One aspect of the method includes one or more optional “quenching” steps. Typically a quenching step is performed to deactivate reactive functional groups associated with an organic thinfilm. In working embodiments cysteine was used to quench thiol-reactive functional groups associated with a thinfilm. In other working embodiments glycine was used to quench amino-reactive functional groups associated with a thinfilm. Suitable quenching agents and procedures can be selected as is known to those of ordinary skill in the art, however, typically the quenching reagent can be selected so that it has a functional group having a similar reactivity as a functional group used to associate a reagent or affinity agent to an organic thinfilm.

In one embodiment of an array of affinity agents, an array of specifically oriented immobilized antibodies is provided. The antibodies are immobilized by conjugating a biotin molecule to the antibody. In one example the antibody is conjugated to the biotin molecule by first oxidizing a vicinal diol functionality on an antibody glycosyl moiety to form the corresponding aldehydes. The glycosyl moiety aldehydes are reacted with a biotin molecule functionalized with an aminooxy group. The reaction yields an antibody-biotin compound covalently bound through an oxime bond. Finally, the antibody-biotin compound is immobilized to an organic thinfilm comprising a streptavidin compound.

Additional reagents and functional groups useful for assembling monolayers and conjugating affinity reagents to organic thinfilms can be selected from those disclosed by Hermanson, G. T. Bioconjugate Techniques, Academic Press: San Diego, 1996, which is incorporated herein by reference.

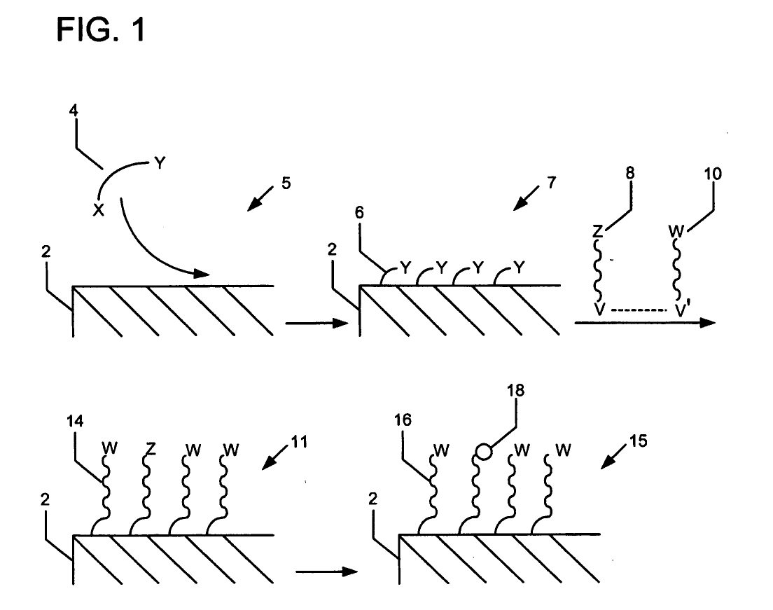

One disclosed embodiment for derivatizing substrates is illustrated in FIG. 1. With reference to FIG. 1, substrate 2 is functionalized with reagent 4 having at least two functional groups X and Y.

Classes of materials useful as substrate 2 for forming arrays as disclosed herein include inorganic materials, metals, and organic polymers. Examples of inorganic materials include silicon, silica, quartz, glass, controlled pore glass, carbon, alumina, titanium oxide, tantalum oxide, indium tin oxide, germanium, silicon nitride, gallium arsenide, zeolites, mica and combinations thereof. Useful metals include aluminum, copper, gold, platinum, titanium, alloys thereof, and combinations thereof. Examples of useful polymers include, without limitation, polyethylene, polyethyleneimine, polyvinylethylene, polystyrene, poly(tetra)fluoroethylene, polycarbonate, polymethylmethacrylate, polydimethylsiloxane, polyvinylphenol, polyoxymethylene, polymethacrylamide, polyimide and copolymers of such materials. Optionally, the substrate coated with one or more different materials, which typically are selected from those listed above. For example, the coating optionally may be a metal film. Possible metal films include aluminum, chromium, titanium, tantalum, nickel, stainless steel, zinc, lead, iron, copper, magnesium, manganese, cadmium, tungsten, cobalt, and alloys or oxides thereof. In an exemplary embodiment, the metal film is a noble metal film. Noble metals that may be used for a coating include, but are not limited to, gold, platinum, silver, and copper. Electron-beam or ion beam evaporation may be used to provide a thin coating of gold on the surface of the substrate. In another exemplary embodiment, the coating comprises an alloy of a noble metal. The metal film may have a thickness ranging from about 50 nm to about 1,000 nm, more particularly from about 100 nm to about 500 nm in thickness. In alternative embodiments, the coating can include silicon, silicon oxide, silicon nitride, titanium oxide, tantalum oxide, silicon nitride, silicon hydride, indium tin oxide, magnesium oxide, alumina, glass, hydroxylated surfaces, and polymers.

X can be any group that yields chemisorption or physisorption of reagent 4 on substrate 2, thereby forming a monolayer. X typically is a group that reacts chemically with substrate 2, and thus X is selected to be compatible with the substrate material. For example, when the substrate is an oxide, such as silicon oxide, indium tin oxide, magnesium oxide, alumina, quartz, glass, or is a material such as silicon or aluminum having an oxidized surface layer, silanes and siloxanes are useful groups for functionalizing the substrate. For example, halosilanes, including mono-, di- and tri-halosilanes can be used to attach a reagent to such substrates. Useful siloxanes that can be used to attach functional groups to the substrate include mono-, di-, and tri-, alkoxysilanes. In working embodiments X was a siloxane, such as a triethoxysiloxane, when substrate 2 was silicon oxide.

Examples of suitable X groups for derivatizing metal substrates or a metal coating on a substrate include those sulfur-bearing compounds, such as thiols, thioethers (sulfides), isothiocyanates, xanthanates, thioacids, thiocarbamate, disulfides (including symmetric and asymmetric disulfides), dithioacids (including symmetric and asymmetric dithioacids) and sulfur-containing heterocycles; selenium bearing molecules, such as selenols, selenides and diselenides (including symmetric and asymmetric diselenides; nitrogen-bearing compounds, such as 1°, 2° and perhaps 3° amines, aminooxides, pyridines, isocyanates, isonitriles, nitriles, and hydroxamic acids; phosphorus-bearing compounds, such as phosphines; and oxygen-bearing compounds, such as carboxylates, hydroxyl-bearing compounds, such as alcohols, and mixtures thereof.

Sulfur-containing compounds are particularly useful for functionalizing silver, gold and platinum surfaces. When the substrate is a metal, such as silver, gold or platinum, thiols and disulfides are preferred reagents for functionalizing the substrate. In working embodiments, thiols and disulfides were used to attach various reagents to gold surfaces

In other embodiments, the surface of the substrate (or coating thereon) is composed of a metal oxide such as titanium oxide, tantalum oxide, indium tin oxide, magnesium oxide, or alumina and X is a carboxylic acid. Alternatively, if the surface of the substrate (or coating thereon) of the device is copper, then X typically is a hydroxamic acid.

If the substrate used in the invention is a polymer, then in many cases a coating on the substrate such as a copper coating will be included in the device. An appropriate functional group X for the coating would then be chosen for use in the device. In an alternative embodiment comprising a polymer substrate, the surface of the polymer may be plasma-modified to expose desirable surface functionalities for monolayer formation. For instance, European Patent Publication No. 780423 describes using a monolayer molecule that has an alkene X functionality on a plasma exposed surface. Still another possibility for the invention device comprised of a polymer is that the surface of the polymer on which the monolayer is formed is functionalized due to copolymerization of appropriately functionalized precursor molecules.

Another possibility is that prior to incorporation into the monolayer, X can be a free-radical-producing or free-radical-activated moiety. Such a functional group is especially appropriate when the surface on which the monolayer is formed is a hydrogenated silicon surface. Possible free radical producing moieties include, but are not limited to, diacylperoxides, peroxides, and azo compounds. Alternatively, unsaturated moieties, such as unsubstituted alkenes (particularly α-β unsaturated ketones), alkynes, cyano compounds and isonitrile compounds can be used for X as free radical activated moieties, particularly if the reaction with X is accompanied by ultraviolet, infrared, visible, or microwave radiation.

In alternative embodiments, X can be a vinyl, sulfonyl, phosphoryl or silicon hydride group.

The linker group in reagent 4 between groups X and Y can be any suitable, chemically compatible spacer group. In one embodiment the linker group is selected to promote the self assembly of reagent 4 into monolayer 6, and/or to ensure that the monolayer is ordered and densely packed. The factors that contribute to self assembly are known to those of ordinary skill in the art, as described by Laibinis, et al. Science 1989, 245, 845 and Ulman, An Introduction to Ultrathin Organic Films: From Langmuir-Blodgett to Self-Assembly, Academic Press (1991).

Typically, the linker group in reagent 4 between functional groups X and Y is a hydrocarbon chain, optionally branched and/or including heteroatoms, such as oxygen. Where the linker group is a hydrocarbon chain, the group typically includes from 2 to about 22 carbon atoms. The term “hydrocarbon chain” as used herein therefore typically refers to a chain of carbon atoms, typically comprising from 2 to about 22 carbon atoms. The chain can comprise aliphatic and aryl groups and can comprise straight chain, branched chain and/or cyclic groups. In one working embodiment the linker group between X and Y was a three carbon chain, and in another working embodiment the linker group was an unbranched 11 carbon hydrocarbon chain.

In one aspect, regardless of the nature of the monolayer molecules, it may be desirable to provide crosslinking between component molecules of a monolayer. In this case, the linker group in reagent can include the functional groups to facilitate crosslinking. In general, crosslinking confers additional stability to the monolayer. Such methods are familiar to those of ordinary skill in the art (for instance, see Ulman, An Introduction to Ultrathin Organic Films: From Langmuir-Blodgett to Self-Assembly, Academic Press (1991)).

With continued reference to FIG. 1, functionalization of substrate 12 with reagent 4 yields monolayer 6 formed on substrate 2, which is further derivatized with reagents 8 and 10 to yield monolayer 14. Thus, the component, Y, of monolayer 6 is a functional group responsible for attaching a second set of reagents, e.g. 8 and 10. Y can function to form a covalent bond with reagents 8 and 10 or can interact with 8 and 10 non-covalently so that 8 and 10 are operatively associated with monolayer 6. Optionally, a reversible covalent bond can be formed so that reagent 8, 10, or both can be dissociated from monolayer 6.

Where 8 and/or 10 are to be covalently attached to monolayer 6, thereby forming a new monolayer, Y typically is either reactive with functional groups V and V′ on reagents 8 and 10, respectively, or alternatively is readily activated for reaction with V and V′.

Examples of functional groups suitable for use as Y include electrophiles, such as activated carboxylic acids, including, for example, acyl imidazolides, acyl azides, and activated esters, such as pentafluorophenol, p-nitrophenol, N-hydroxysuccinimide and sulfo-N-hydroxysuccinimide esters. Additional useful electrophiles include Michael acceptors, such as α,β-unsaturated carbonyl groups (including acids, amides, esters, ketones and aldehydes), α,β-unsaturated sulfoxides, α,β-unsaturated sulfones, and the like. Preferred Michael acceptors include maleimide and maleimide derivatives. Additional examples of electrophiles useful as Y groups include aldehydes and alkylating agents, such as epoxides, aziridines, and alkyl halides, such as alkyl iodides, bromides and chlorides, particularly α-haloacetyl groups and primary and tertiary alkyl iodides, bromides and chlorides. Similarly, alkylating agents such as alkyl sulfonic acid esters also are useful alkylating agents, with specific examples including methylenesulfonic acid, trifluoromethylenesulfonic acid and p-toluenesulfonic acid esters.

Additional examples of functional groups suitable for use as Y include nucleophilic groups, such as hydroxyl, peroxide, peroxyacid, sulfhydryl, thioacid, selenol, amino, aminooxy, hydrazide and thiosemicarbazide groups.

Another class of reactive Y group that can be used includes functional groups that engage in cycloaddition reactions. Examples of useful cycloaddition reactions include, without limitation 2+2, 3+2, particularly 1,3-dipolar cycloadditions, and 4+2 cycloaddition reactions. For example, Y can be a dienophile used in a Diels-Alder reaction. Examples of suitable dienophiles include electron poor alkenes or, alternatively, in an inverse-demand Diels-Alder reaction, electron-rich alkenes. Conversely, electron-rich dienes or electron-poor dienes are used in Diels-Alder and inverse-demand Diels-Alder cycloaddition reactions, respectively.

Azide-containing compounds can be used in several different types of cycloaddition reactions. For example, 1,3-dipolar cycloaddition reactions can be used to link a compound to monolayer 6 when Y includes an azide, which can be reacted with a suitable alkene compound to yield, following thermal elimination of nitrogen, the corresponding aziridine compound. Conversely, Y can include an alkene group that can react with an azide-containing compound.

Azides also can be used to react with an in situ-formed ketene in Staudinger-type cycloadditions, which yields a β-lactam-containing product. Suitable precursors and conditions for using cycloaddition reactions to functionalize monolayer 6 can be selected by those of ordinary skill in the art.

Another class of reaction that can be used to functionalize monolayer 6 covalently includes organometallic reactions. Specific organometallic reactions include, without limitation, metathesis reactions, for example, in one embodiment an aldehyde- or alkene-functionalized surface can be reacted with reagents 8 and 10 wherein V and V′ include an aldehyde or alkene functional group. Examples of catalysts and conditions for such metathesis reactions are known to those of ordinary skill in the art. Certain examples of such catalysts include the metals titanium, tungsten, molybdenum or ruthenium. See, for example, Ivin, K. J., Mol, J. C. Olefin Metathesis and Metathesis Polymerization; Academic Press: London, 1997.

Where Y is a functional group that is activated in situ, possibilities for Y include, but are not limited to, moieties such as an azide, hydroxyl, carboxyl, amino, aldehyde, carbonyl, methyl, methylene, alkene, alkyne, carbonate, aryliodide, or a vinyl group. Appropriate modes of activation of such functional groups are known to those of ordinary skill in the art. For example, where Y is a carboxyl group, Y can be activated in situ for acylation by, for example, converting Y into an activated ester, such as a pentafluorophenol, p-nitrophenol, N-hydroxysuccinimide or sulfo-N-hydroxysuccinimide ester. Moreover, such activated esters react readily with a variety of suitable nucleophiles, such as amines, thiols, alcohols, and the like. In one aspect, Y may be a protected functional group that can be removed in situ to unmask a reactive functional group. Suitable protecting groups that can be used can be selected, installed and removed as is known to those of ordinary skill in the art, and include protecting groups disclosed in Greene, T. W.; Wuts P. G. M. Protective Groups in Organic Synthesis, 3rd ed.; Wiley-Interscience, 2002. In a working embodiment a protected aldehyde was used to produce a masked aldehyde-functionalized substrate.

In an alternative embodiment, the functional group Y of the monolayer is selected from the group of charged moieties. Possible charged Y functional groups include, but are not limited to, phosphate, sulfate, carboxylate, and the like. Simple groups such as these can be used for Y when reagent 8, 10, or both include a ionic group, such as poly-lysine, that coats the exposed portion of the monolayer.

With continued reference to FIG. 1, reagents 8 and 10 are attached to monolayer 6, thereby forming new, second monolayer 14, which includes the components of first monolayer 6. Reagent 8 has first functional group V for attachment to monolayer 6 and second functional group Z, which can be used, for example, to attach an affinity reagent. Reagent 10 includes functional group V′ for attachment to monolayer 6 and also includes a protein resistant component W. Functional groups V and V′ can be the same or different functional groups. Optionally, reagents 8 and 10 can be delivered in two different steps. Thus, reagent 8 could be deposited either before or after reagent 10. In one embodiment V and V′ are optionally covalently linked in, for example, a disulfide bond, such that reagents 8 and 10 having functional groups Z and W, respectively, are delivered in the same step. Functional groups V and V′ are selected with reference to functional group Y displayed on monolayer 6. As noted above, with respect to functional group Y, V and/or V′ can form a covalent linkage to monolayer 6, or can associate non-covalently with monolayer 6. Generally, V and V′ are selected to be complementary to functional group Y, thus, for example, where Y is an electrophilic group, V and V′ typically are nucleophilic groups, and conversely, where Y is a nucleophilic group, V and V′ typically are electrophilic groups.

Functional group Z on monolayer 14 is used to attach an affinity agent to yield monolayer 16 having affinity agent 18 bound thereto.

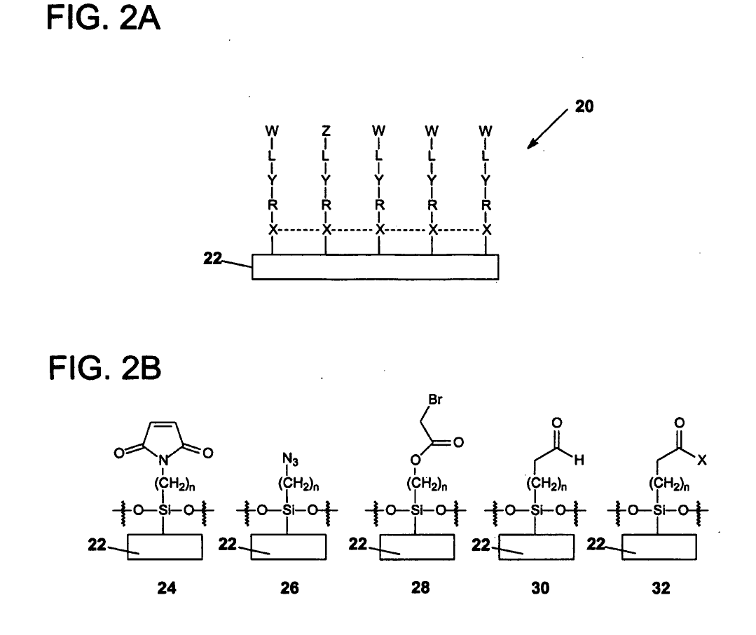

FIG. 2 includes a schematic representation 20 of arrays disclosed herein and also depicts several exemplary reactive monolayer functional groups. With reference to array 20, substrate 22 can be any suitable substrate. X represents a functional group for non-covalently or covalently (reversibly or irreversibly) associating a reagent with substrate 22. Optionally, one or more functional groups X depicted in array 20 are linked directly or indirectly to one another. For example, groups X can be linked before association with the substrate 22 by a polymer backbone, such as a poly-L-lysine backbone. Alternatively, X can be a group (or derived from a group) such as a siloxane, which can react with one or more during or after association with substrate 22.

R represents a linker group, such as a linker group of the type discussed with respect to reagent 4 above. Typically, R is a substantially unbranched chain, such as an unbranched hydrocarbon chain. Where R includes a hydrocarbon chain, the chain typically has from 2 to about 22 carbon atoms, and more typically R has from 3 to about 18 carbon atoms. In working embodiments the various hydrocarbon chains used included those having 3, 4, 10, 11, and 12 carbon atoms.

Y represents a linkage, typically formed as disclosed herein when a second reagent is associates covalently or noncovalently with a substrate-associated monolayer. Where Y represents a covalent linkage, Y is typically formed by the reaction of two functional groups where one can be described as electrophilic and the second as nucleophilic. Useful examples of electrophilic and nucleophilic groups include those discussed above, with respect to FIG. 1. Other functional groups that can react to form Y, and that typically are not characterized as either nucleophilic or electrophilic, include functional groups that engage in cycloaddition and organometallic reactions.

With continued reference to FIG. 2, structures 24, 26, 28, 30 and 32 depict representative monolayer functional groups used in working embodiments to incorporate at least a second reagent into a monolayer formed on a glass or silicon substrate. For example, the maleimide functional group in structure 24 can be used as an electrophile in a Michael reaction, or, alternatively can be used in a cycloaddition reaction, such as a Diels-Alder reaction.

The azide moiety in structure 26 can be activated to function as a nucleophile via reduction to the corresponding amine, or can be used in a Staudinger ligation reaction, which accomplishes activation, reduction and acylation in a single step in situ. Alternatively the azide functionality can be used directly in a cycloaddition reaction to react with, for example an in situ-formed ketene reagent.

Structure 28 includes a bromoacetate moiety that is an excellent electrophile, with the bromine being subject to facile nucleophilic displacement by a variety of nucleophiles, particularly thiolates. With reference to structure 28, X can be a nitrogen or an oxygen.

Structure 30 includes an aldehyde, which can be used as an electrophile for reaction with a various nucleophilic reagents. A variety of nucleophilic groups capable of reacting with an aldehyde to produce a covalent bond are useful in the current invention. Useful reactive groups include, for example, alkoxides, enolates, carbanion equivalents, such as Grignard-type reactive groups, alcohol groups (yielding the corresponding acetal or hemiacetal), cyanide reactive groups, amines, including primary or secondary amines, hydrazines, phosphorus ylides, phosphine oxide anions, aminooxy reagents, hydrazides thiosemicarbazides, and the like. Alternatively, an aldehyde can be used in an organometallic reaction, such as an aldehyde-alkene metathesis reaction.

Structure 32 represents an acylating group, such as an activated ester. Thus, X is a group that is readily displaced, as is known to those of ordinary skill in the art, such as a halide, N-hydroxysuccinimide, sulfo-N-hydroxysuccinimide, pentafluorophenol, p-nitrophenol, and the like.

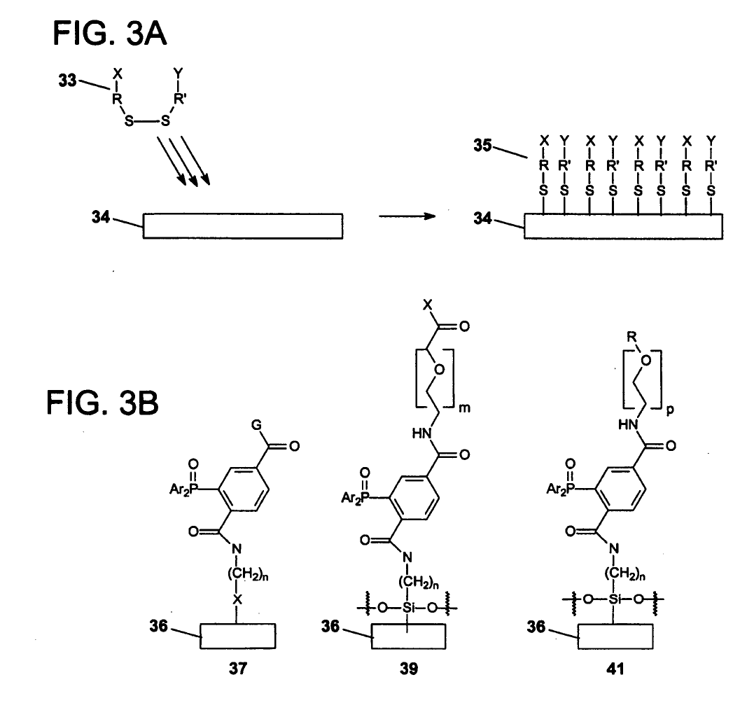

FIG. 3 illustrates a method for preparing organic thinfilm arrays by first derivatizing a substrate 34 using an asymmetric disulfide 33. The disulfide 33 is asymmetric in the sense that it is composed of two different substituents. Deposition according to this method yields a monolayer 35, which includes two different monolayer components. Optionally, one or more additional symmetric or asymmetric disulfides can be deposited to provide a monolayer including three or more different monolayer components, or simply a different ratio of the two monolayer components provided by the asymmetric disulfide. For example, where a three-to-one ratio of X to Y groups is desired, deposition of asymmetric disulfide 33 can be accompanied by deposition of an equal amount of a symmetric disulfide having the formula X—R—S—S—R—X. X and Y in monolayer 35 function as an affinity agent and a protein resistant component or as reactive functional groups for incorporating an affinity agent and protein resistant reagent. Optionally, where X and Y are such reactive functional groups, they can be the same or different functional groups.

FIG. 3 also illustrates representative monolayer components formed by a Staudinger ligation method as disclosed herein. With reference to structure 37, X is a functional group suitable for associating a monolayer or organic thinfilm with substrate 36. X typically is attached to a hydrocarbon linking group as illustrated in structure 37; however, other linking groups also can be used in such structures. Typically, the selected hydrocarbon chains have from 2 to about 22 carbon atoms, and thus ‘n’ is from 2 to about 22. More typically n is from 3 to about 18. G can be any group useful for covalently or non-covalently associating an affinity agent or protein resistant component, or another reactive functional group, which can be used to covalently or non-covalently associate an affinity agent or protein resistant reagent.

Referring again to FIG. 3, structures 39 and 41 represent monolayer components formed on a substrate 36. With reference to structure 39, substrate 36 is functionalized with a siloxane functional group linked to a hydrocarbon chain. The illustrated hydrocarbon chain includes ‘n’ methylene groups, where n typically is from 2 to about 22, and more typically is from 3 to about 18. In one working embodiment n was 11. The hydrocarbon chain is functionalized with an aminoacyl aryl phosphine oxide moiety, which is installed via a Staudinger ligation protocol. At the para position relative to the hydrocarbon amino acyl group is an acyl amino group, bonded to a polyethylene glycol chain comprising ‘m’ ethylene glycol units. The ethylene glycol chain can be any length, however typically m is from 2 to about 100 and more typically from 2 to about 70 ethylene glycol units. Most typically m is from about 2 to about 20. One working example included monolayer components according to structure 39 where m was 6.

With continued reference to structure 39, X is a group that renders the monolayer component an active acylating agent. Such acylating agents can be prepared from the corresponding carboxy group as is known to those of ordinary skill in the art. Typically, 39 is an activated ester group, such that X is a pentafluorophenol, p-nitrophenol, N-hydroxysuccinimide, sulfo-N-hydroxysuccinimide, or the like. Additional suitable X groups include halides, imidazolides, anhydrides, and the like. Structure 41, like structure 39, illustrates a monolayer component formed on a glass or silicon substrate. However, 41 functions as a protein resistant component. With reference to 41 ‘p’ is from 2 to about 100 ethylene glycol units and more typically from 2 to about 70 ethylene glycol units. Most typically, ‘p’ is from 2 to about 20 ethylene glycol units. R represents an alkyl group, hydrogen or a hydroxyl bearing group. Typically, R is either a methyl group or a hydrogen.

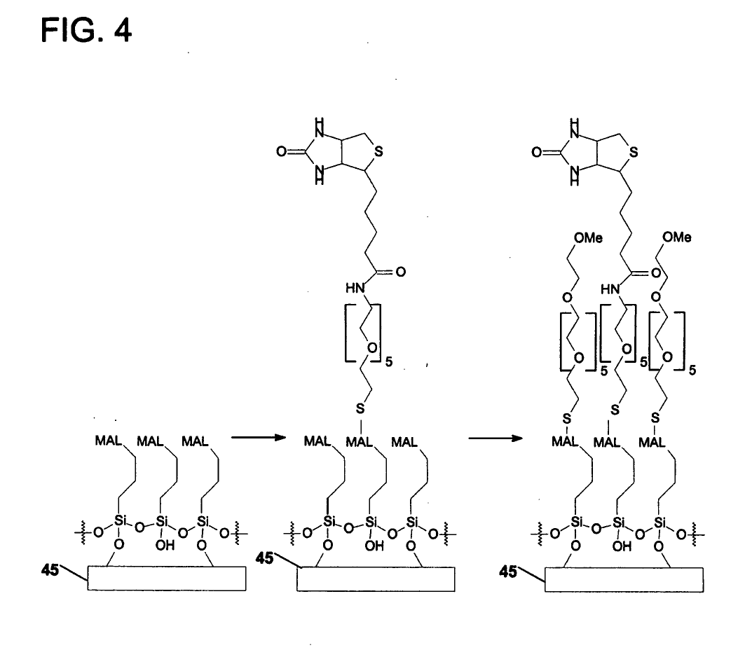

FIG. 4 illustrates another embodiment for functionalizing a substrate. In this embodiment, substrate 45 is glass or has a silicon oxide coating, which is derivatized with a 3-maleimidopropyl-1-silane derivative to give the monolayer coated substrate shown having a maleimide (MAL) functional group on one face. The maleimide group can be used to attach a sulfhydryl-containing reagent with high efficiency. In the illustrated example a heterobifunctional reagent having a sulfhydryl group at a first end and a biotin group at a second end is attached to the substrate via the maleimide group. A reagent containing a protein-resistant component is then attached via remaining maleimide functional groups.

### D. Uses for the Disclosed Arrays

The disclosed arrays are useful for characterizing the interaction of soluble analytes with arrayed affinity agents. In particular, the arrays can be used to identify affinity agents that bind to a molecule of interest, particularly a biomolecule of interest, such as a protein or nucleic acid. The arrays are especially useful for high throughput drug screening, using both small molecule and biomolecule arrays.

A wide range of detection methods is applicable to characterizing the interactions of soluble analytes with the disclosed arrayed affinity agents. For example the arrays can be incorporated into a device that is interfaced with optical detection methods such as absorption in the visible range, chemoluminescence, and fluorescence (including lifetime, polarization, fluorescence correlation spectroscopy (FCS), and fluorescence-resonance energy transfer (FRET)). Furthermore, built-in detectors such as optical waveguides as disclosed in PCT Publication WO 96/26432 and U.S. Pat. No. 5,677,196, incorporated herein by reference, surface plasmon resonance, and surface charge sensors are compatible with embodiments of the arrays disclosed herein.

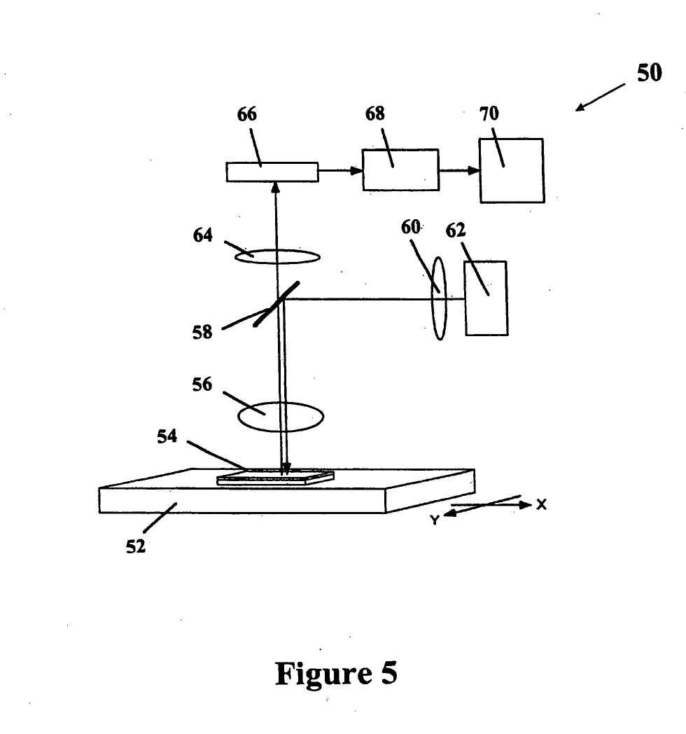

An exemplary type of fluorescence detection unit that may be used to monitor interaction of immobilized affinity agents of an array with an analyte is described in U.S. Pat. No. 6,406,921 to Wagner et al. (Wagner), which is incorporated herein by reference in its entirety. FIG. 5 is a schematic diagram of this type of fluorescence detection unit, which may be used to monitor interaction of immobilized affinity agents of an array with an analyte. In the illustrated detection unit, the array 54 is positioned on a base plate 52. Light from a 100 W mercury arc lamp 62 is directed through an excitation filter 60 and onto a beam splitter 58. The light is then directed through a lens 56, such as a Micro Nikkor 55 mm 1:2:8 lens, and onto the array 54. Fluorescence emission from the array returns through the lens 56 and the beam splitter 58. After next passing through an emission filter 64, the emission is received by a cooled CCD camera 66, such as the Slowscan TE/CCD-1024SF&SB (Princeton Instruments). The camera is operably connected to a CPU 68, which is in turn operably connected to a VCR/monitor 70.

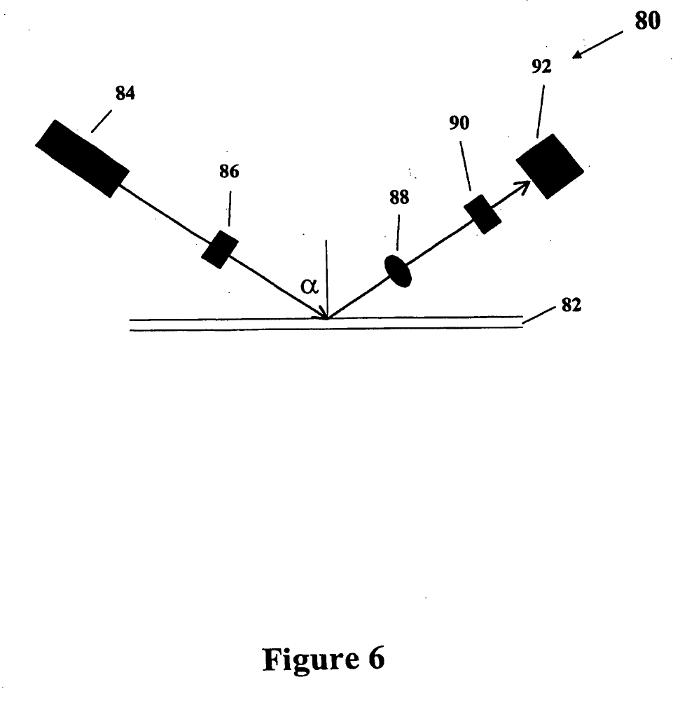

FIG. 6 is a schematic diagram of an alternative detection method based on ellipsometry. Ellipsometry allows information about the sample to be determined from the observed change in the polarization state of a reflected light wave. Interaction of an analyte with a layer of immobilized affinity agents on a substrate results in a thickness change and alters the polarization status of a plane-polarized light beam reflected off the surface. This process can be monitored in situ from aqueous phase and, if desired, in imaging mode. With reference to FIG. 6, in a typical setup 80, monochromatic light (e.g. from a He—Ne laser, 84) is plane polarized (polarizer 86) and directed onto the surface of the sample on substrate 82 and detected by a detector 92. A compensator 88 changes the elliptically polarized reflected beam to plane-polarized. The corresponding angle is determined by an analyzer 90 and then translated into the ellipsometric parameters Psi and Delta which change upon binding of analyte with the immobilized proteins. Additional information can be found in Azzam, et al., Ellipsometry and Polarized Light, North-Holland Publishing Company: Amsterdam, 1977.

### E. EXAMPLES

The foregoing disclosure is further explained by the following non-limiting examples. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature and pressure is at or near atmospheric.

### General Methods

Glass and silicon substrate materials were cleaned prior to derivatization according to the following procedure: 200 mL of 30% H₂O₂ (Aldrich, Milwaukee, Wis.) and 200 mL concentrated NH₄OH (Aldrich) were added to a 65° C. bath containing 1000 mL of nanopure water (18 MΩ resistance). A Teflon rack holding glass microscope slides (1″ by 3″, Fisher Scientific) or 1 cm×2 cm silicon wafers was immersed in the cleaning bath. After about 15 minutes, the slide rack was removed from the bath and placed into a 300 mL bath of nanopure water for 10 minutes with gentle agitation, followed by immersion in a second 300 mL bath of nanopure water for 10 minutes with gentle agitation. The slides were stored under nanopure water and dried immediately before use via spin rinse drying, using a cycle of 3 minutes washing at 600 rpm, 10 seconds nitrogen purging at 1500 rpm, followed by two drying cycles of 1.5 and 20 minutes at 2500 and 600 rpm, respectively.

### Example 1

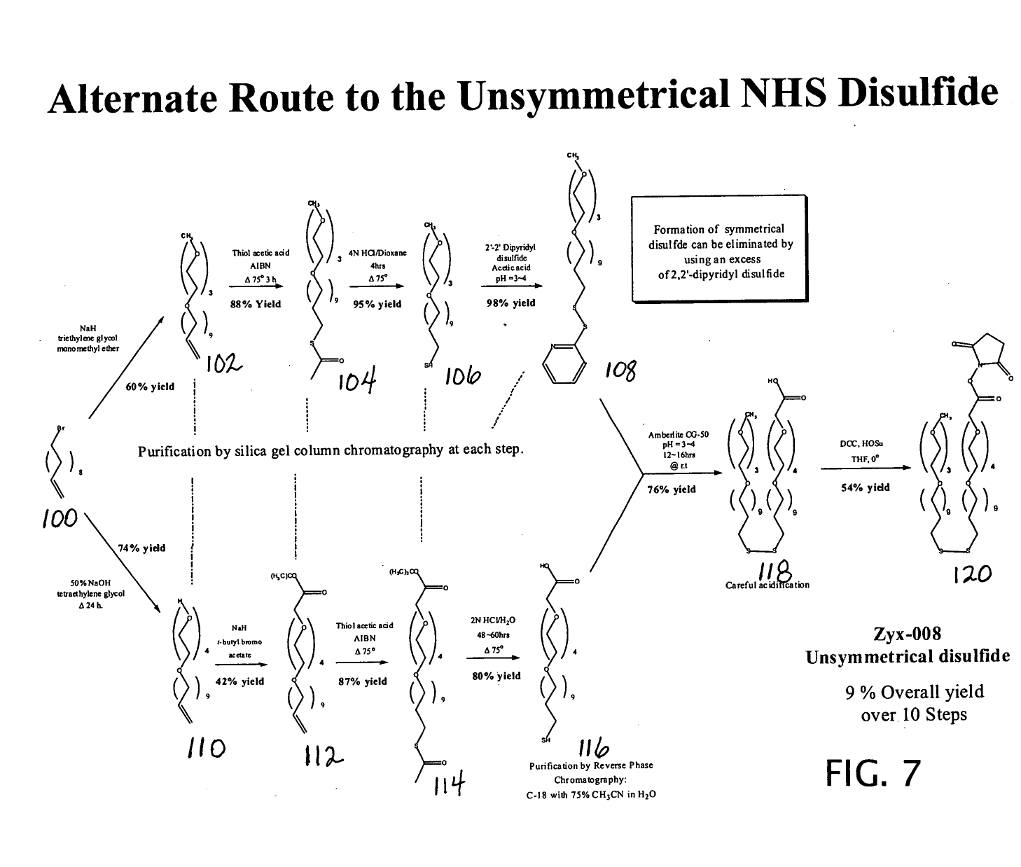

This example describes the formation of an aminoreactive organic thin film on a gold substrate using disulfide thin film precursors. A freshly prepared Au(111) chip was immersed in a 1 mM solution of asymmetric disulfide compound 120 (the synthetic route to compound 120 is illustrated in FIG. 7 and described in detail below) at room temperature for 1 hour. The chip was then rinsed with ethanol (3×10 mL). After rinsing, the coated chip was dried under a nitrogen stream and used immediately for reaction with an amine-containing reagent.

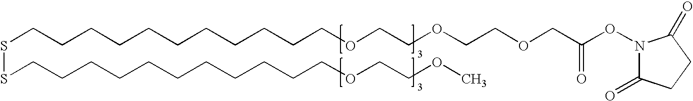

### Example 2

This example describes functionalizing an aminoreactive organic thin film to form a biotin-functionalized chip. A chip having an aminoreactive coating prepared according to example 1 was incubated with a 5 mM solution of either (+)-biotinyl-3,6-dioxaoctanediamine or (+)-biotinyl-3,6,9,11-tetraoxatridecanediamine (commercially available from Pierce, Rockford, Ill. and Molecular BioSciences, Boulder, Colo., respectively) in 50 mM phosphate buffer, pH 7.5 containing 0.05% Tween-20 (polyoxyethylene(20) sorbitan monolaurate) commercially available from Sigma-Aldrich, Milwaukee, Wis.) for 10 minutes at room temperature. The chip was washed with 500 mM ionic strength PBS buffer, pH 7.5 (total wash volume 30 mL). The chip was then immersed in a 37° C. 1M glycine solution to deactivate any remaining aminoreactive NHS groups.

### Example 3

This example describes the general protocol used for silanization of silicon oxide substrates. Reagents deposited by this method included 11-azidoundecyl-1-triethoxysilane and 3-maleimidopropyl-1-triethoxysilane, which was prepared according to the protocol of Shaltout et al. Mat Res Soc. Symp. Proc. 1999, 576, 15-20, which is incorporated herein by reference. The Shaltout procedure also was applied to the synthesis of other maleimide derivatives. 3-Maleimidopropyl-1-triethoxysilane reagent was deposited as follows: To a clean, dry 120 mL PTFE vial (Savillex, Minnetonka, Minn.) was added 50 mL dry toluene (99.8% anhydrous, Aldrich catalog number, 24,451-1, Milwaukee, Wis.) and 1 mL hexanoic acid (Aldrich catalog number 15374-5), followed by 0.5 mL of neat silane. The vial was swirled and dry, clean silicon oxide wafers were added to the silane solution. The silanization was allowed to proceed for 24 hours at room temperature on a shaker (ca. 100-150 rpm). The wafers were rinsed with dry toluene and absolute ethanol and then sonicated under absolute ethanol for 10 minutes. The sonicated wafers were dried under nitrogen and stored in a fluoroware container in a dessicator under argon. The silanized chips can be used for about 2 to 3 weeks.

### Example 4

This example describes the general Staudinger ligation protocol used to couple aminoreactive and protein resistant components to the 11-azidoundecyl-1-triethoxysilane functionalized substrate prepared according to the protocol of example 3. The protein resistant Staudinger reagent 20 and aminoreactive Staudinger reagent 30 are depicted below. Staudinger reagents 20 and 30 were prepared according to the method disclosed by Saxon and Bertozzi in U.S. Pat. No. 6,570,040, which is incorporated herein by reference in its entirety. 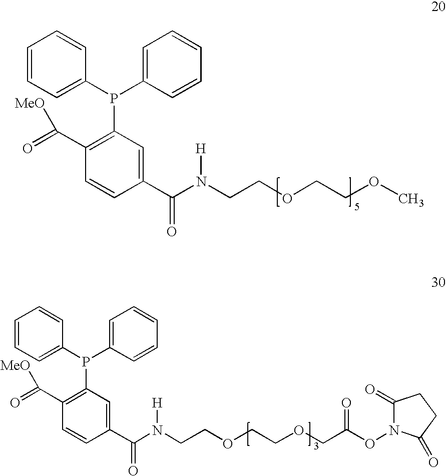
Staudinger reagent was dissolved in 4 mL of oxygen purged (via nitrogen sparging) 3:1 tetrahydrofuran:nanopure (deionized, 18 MΩ) water to give a concentration of 20 mM. The 11-azidoundecyl-1-triethoxysilane chip was placed in a teflon container and was immersed in the Staudinger reagent solution. The container was shaken on a shaker for 24 hours at room temperature, and then the chip was removed and rinsed with 30 mL each of 3:1 tetrahydrofuran:nanopure water, nanopure water, and absolute ethanol. Each chip was dried under nitrogen and stored until further use. The progress of the Staudinger ligation can be monitored by IR spectroscopy, and the yield can be determined by quantitative IR spectroscopy.

### Example 5

This example describes the functionalization of a 3-maleimidopropyl-1-triethoxysilane substrate with a biotin derivative 40 and a protein resistant component 50. A maleimide functionalized chip prepared according the protocol of example 3 was coated with 250 μL of an 80 μM solution of compound 40 in buffer (20 mM citrate, 150 mM sodium, 5 mM EDTA, 0.05% Tween 20 buffer at pH 6.5). The chip was incubated at room temperature for 30 minutes with slight agitation on a shaker. The chip was washed with buffer (0.5 M sodium in 1×PBS, 0.05% Tween 20, pH 7.5), and then incubated with 150 μL an 80 μM solution of compound 50 in buffer (20 mM citrate, 150 mM sodium, 5 mM EDTA, 0.05% Tween 20 buffer at pH 6.5) at room temperature for 30 minutes with slight agitation. The chip was washed with buffer (0.5 M sodium in 1×PBS, 0.05% Tween 20, pH 7.5), and then immersed in a cysteine quenching solution (0.1M cysteine, 20 mM citrate, 150 mM sodium, 5 mM EDTA, 1 mM tris-(2-carboxyethyl)phosphine hydrochloride (TCEP), 0.05% Tween 20, pH 6.5) for 30 minutes at 37° C. The quenched chip was washed with 30 mL of buffer (0.5 M sodium in 1×PBS, 0.05% Tween 20, pH 7.5) and 30 mL of nanopure water. The chip was spin dried and used immediately. The biotin functionalized chips prepared according to this protocol can be stored under argon at 4° C. for up to about 3 weeks.

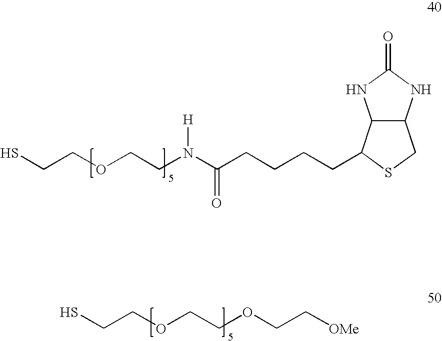

The biotin functionalized surfaces prepared as described above in example 5 can be used to immobilize different biotin-derivatized reagents on the surface via mutual streptavidin binding. The following examples describe four different antibody immobilization strategies. The four types are: randomly biotinylated IgG; oriented IgG (biotinylated on carbohydrate on Fc domain); oriented Fab′ fragments (biotinylated in hinge region) and randomly biotinylated Fab′ fragments. Methods for preparing these biotinylated antibodies are described below.

### Example 6

This example describes the preparation of an aldehyde functionalized organic thinfilm. Ethylene glycol (28 mL, 31.4 g, 505 mmol, 1.05 equiv.) was added to a 1 L round bottom flask containing undecylenic aldehyde 60, toluene (500 mL), followed by p-toluenesulfonic acid hydrate (13.7 g, 72 mmol, 0.15 equiv.) was added and the solution heated to 125° C. The solution was refluxed under a Dean-Stark trap for 3 hours. After approximately 13 mL of water was collected in the trap, the solution was allowed to cool to room temperature, was diluted with toluene, and washed with aqueous sodium bicarbonate solution (1N, 2×300 mL) and brine (ca. 400 mL). The organic solution was dried over Na₂SO₄ and concentrated in vacuo to a brown oil. The crude material was purified by distillation at 85-88° C. and 48 mTorr. Compound 70 was isolated as clear liquid (approximately 110 mL). ¹H NMR (400 MHz, CDCl₃): δ 1.3 (m, 12H), 1.6 (m, 2H), 2.0 (m, 2H), 3.85 (AA′BB′, 2H), 3.95 (AA′BB′, 2H), 4.85 )t, 1H), 4.9-5.0 (two d, 2H), 5.8 (ddt, 1H). 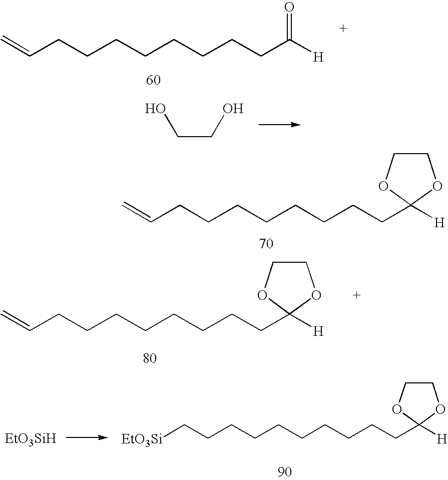
11-triethoxysilylundecyl ethylene glycol acetal 90 was prepared from alkene acetal 70 (38 mL, 34.2 g, 161 mmol) was added to a 100 mL round bottom flask. Pt(Ph₃P)₄ (0.20 g, 0.161 mmol, 0.001 equivalent)was added, followed by Pt octanal/octanol (2.5 Pt solution, 1.25 mL, 0.161 mmol, 0.001 equivalent). Triethoxysilane (29.75 mL, 26.5 g, 161 mmol, 1.0 equivalent) was added to the alkene catalyst solution at ambient temperature, resulting in a slight exotherm. The solution was stirred at ambient temperature for 1.5 hours, and then the reaction was heated to 105° C. for two hours. Completion of the reaction was determined by monitoring the disappearance of the alkene resonances in the ¹H NMR spectrum. Desired product was obtained by distillation at 114-130° C. and 13 mTorr. A clear, colorless liquid was obtained, yielding 34 mL. ¹H NMR (400 MHz, CDCl₃): δ 0.57 (m, 2H, —CH₂Si—), 1.176 (m, 25H, C—CH₂—C and C—CH₃), 1.35 (m, 2H, —Si—C—CH₂—C), 1.6 (m, 2H, glycol AA′BB′), 2.8 (m, 1H, C—CH(OR). ¹³C NMR (100 MHz, CDCl₃): δ 10.4 (Si—C), 18.2 (OC—CH₃), 22.8, 24.1, 29.3-29.6 multiple resonances, 33.2, 33.9, 58.3, (O—C—CH₃), 64.8 (O—CH₂—CH₂—), 104 (C—CH(OR)—OR).

Silanization was carried out as follows. To a glass Copeland jar, prerinsed with a small amount of dry toluene, was added 50 mL dry toluene and 0.5 mL aldehyde silane. The jar was placed on a shaker to mix the solution and five clean glass microscope slides were placed into the slots inside. After 24 hours of gentle agitation, the solution was decanted and replaced with 50 mL dry toluene and the jar was shaken for five minutes; this procedure was repeated twice. After decanting the final toluene rinse, 50 mL of ethanol was added and the jar was allowed to shake for 5 minutes. After decanting the first ethanol rinse, an additional 50 mL of ethanol was added and the jar sonicated for 10 minutes. Finally, the slides were removed from the Copeland jar and dried under nitrogen. The slides were stored under nitrogen to avoid aldehyde oxidation.

Silicon wafers coated with acetal silane 90 was immersed in a 0.1 M HCl solution and agitated for 0.25, 1, 4, 8 and 16 hours. Upon removal from the acid bath, the wafer was immersed in 1 M pH 7 NaHCO₃ for 1 minute, removed, immersed in water and agitated for 5 minutes and then dried using a stream of nitrogen gas.

The deprotection reaction can be followed by X-ray photoelectron spectroscopy (XPS, 45° C. take off angle). The disappearance of the peak corresponding to the C—O bond at 287 eV can be monitored, and the deprotection reaction is nearly complete at 1 hour. The resulting aldehyde functionalized surface can be derivatized with 3′-aminofunctionalized oligonucleotides via reductive amination. Oligonucleotide, 5 μM, is spotted on the aldehyde functionalized surface, and the surface is allowed to dry. The spotted surface is rinsed in a solution of 0.2% SDS in deionized water for 2 minutes at room temperature with agitation. The surface is then immersed in a fresh reducing solution (1.5 g NaBH₄, 133 mL absolute ethanol, and 450 mL PBS at pH 7.2-7.4) and soaked for 5 minutes at room temperature.

### Example 7

This example describes the preparation of 11-triethoxysilylundecyl-2-bromoacetate. 10-Undecenyl alcohol (100 g, 0.59 mole) and α-bromoacetic acid (90 g, 0.65 mole) were weighed into a 1 L round bottom flask and dissolved in 600 mL of dichloromethane. Dicyclohexylcarbodiimide (DCC, 133.3 g, 0.65 mole) was dissolved in 250 mL of dichloromethane and added in portions to the acid/alcohol mixture. A white precipitate quickly formed and some heat was evolved. The mixture was allowed to stir overnight, and then the precipitate (dicyclohexyl urea, DCU) was removed via filtration. The filtrate was concentrated to an oil and taken up in 500 mL of hexanes to precipitate more DCU, which was removed via filtration. The solution was concentrated to give a colorless oil and the product was distilled (74° C. at 4.0 mTorr) yielding a colorless liquid (115.77 g, 68% yield). ¹H NMR (CDCl₃) δ 1.24-1.44 (m, 14H)1.62 (m, 2H), 2.05 (dd, 2H), 3.79 (s, 2H), 4.20 (t, 2H), 4.93 (m, 2H) 5.79 (dddd, 1H). ¹³C NMR (CDCl₃) δ 25.69, 25.92, 28.36, 28.86, 29.03, 29.11, 29.32, 29.37, 33.74, 66.33, 114.12, 138.97, 167.09.

The undecenyl α-bromo acetate, >97% pure, (20g, 0.07 mole) and 280 mg of hydrogen hexachloplatinate (1 mole % ) were added to a round bottom flask. Triethoxysilane (12.46 g, 76 mmol) was added by syringe under Ar. The mixture was placed in a preheated (90° C.) oil bath. Over the course of the reaction (12 hours), the mixture turns a dark color. The product was purified by distillation, 150° C. at 6.0 mTorr. The product was isolated as a clear colorless liquid (13.6 g, 43.5% yield). ¹H NMR (CDCl₃) δ 0.55 (t, 2H), 1.15-1.45 (m, 17H), 1.60 (m, 2H), 2.05 (m, 2H), 3.79 (m, 10H), 4.15 (t, 2H). Elemental Analysis (Theoretical % C 50.10, % H 8.63, % Si 6.17, % Br 17.54), Found: % C 50.18, % H 9.16, % Si 8.10, % Br 15.31.

Silanation using the bromoacetal silane was performed as with the aldehyde silane (example 6), except that the deposition time was only 2 hours (instead of 24) and that care was taken to keep all jars in the dark. Bromoacetyl silane coated slides were stored in a desiccator in the dark.

### Materials for Example 8-10

MAB9647 is a mouse IgG₁ that was raised against the human IL-8; it was produced by Covance Inc. (Princeton, N.J.) from mouse ascites fluid using the hybrodoma cell line HB-9647 from ATCC (Manassas, Va.); it is protein G-purified. MAB208 is a mouse IgG₁ that was raised against human IL-8 by R&D Systems (Minneapolis, Minn.); it is protein G-purified from mouse ascites fluid, clone number 6217.1 11, catalog number MAB208. MAB602 is a mouse IgG_{2A} that was raised against human IL-2 by R&D Systems; it is protein G-purified from mouse ascites fluid, clone number 5355.111, catalog number MAB602. As a detection antibody for the IL-8 assays, an R-phycoerythrin (PE)-conjugated mouse anti-human IL-8 antibody was used (BD Pharmingen, cat. No. 20795A). For the IL-2 assays, two detection antibodies were used: a goat anti-human IL-2 antibody (R&D, cat. No. AF-202-NA) and an R-PE-conjugated donkey anti-goat IgG F(ab′)2 (Jackson Immuno Research, cat. No. 705-116-147). Unless noted, chemicals were purchased from Sigma-Aldrich (St. Louis, Mo.). Pepsin-agarose was purchased from Pierce (Rockford, Ill.), product number 20343. PNGase F was obtained from New England Biolabs (Beverly, Mass.), product number P0704. Aldehyde-reactive probe was from Molecular Probes (Eugene, Oreg.). Human interleukin-2 (IL-2) was purchased from Leinco Technologies (St. Louis, Mo.), product number 011R455, and was reconstituted in phosphate buffered saline (PBS; 11.9 mM sodium phosphate, 137 mM NaCl, 2.7 mM KCl, pH 7.4). A DNA sequence encoding the mature version of human Interleukin-8 (IL-8; AVLPRSAKELRCQCIKT′YSKPFHPKFIKELRVIESGPHCANTEIIVKLSDGRELCLDPKENWVQRVVEKFLKRAENS) SEQ ID 1 with a C-terminal Factor Xa cleavage site and Protein Kinase A site (GIEGRRRASV) SEQ ID 2 was created by gene assembly of oligonucleotides. This construct was inserted into the NdeI and XhoI sites of pET24a (Novagen, Madison, Wis.), resulting in the further addition of a His(6) tag at the extreme C-terminus (LEHHHHHH SEQ ID 3; where the LE codons comprise the XhoI site). This plasmid was then used to direct expression of IL-8 in BL21 DE3 cells (Novagen) grown in EZMix modified Terrific Broth (Sigma, St. Louis, Mo.; CAT No. T-9179) by the addition of 1 mM IPTG at an OD of 0.6 and growth for four hours in a BioFlo3000 fermentor (New Brunswick Scientific, Edison, N.J.) at 30° C. The cells were collected by centrifugation, resuspended in 5 ml buffer/g with 300 mM NaCl, 50 mM sodium phosphate, 5 mM beta-mercaptoethanol, 5 mM imidazole, pH 8.0 including one Complete Protease Inhibitor Cocktail tablet (Roche Applied Science; Cat No. 1697498) per 50 ml and lysed using a microfluidizer. The soluble fraction of IL-8 was purified by metal affinity chromatography using TALON Superflow beads (Clontech, Palo Alto, Calif.; Cat No. 8908-2) followed by gel filtration in PBS using a Superdex 75 prep grade column (Amersham Biosciences). The IL-8 containing fractions were concentrated to 0.4-0.5 mg/ml using a model 8400 stirred ultrafiltration cell (Millipore, Bedford, Mass.; CAT No 5124) with a 3K MWCO membrane and then dialyzed into PBS with 10% glycerol for storage. Identity and Correct secondary structure was confirmed by mass spectrometry, ELISA and circular dichroism (Spectrapolarimeter J-810, Jasco, Easton Md., www.jascoinc.com).

### Example 8

This example describes the preparation of biotinylated Fab′ fragments. The conserved N-linked glycosylation of IgG's were removed from the antibodies under the following reaction conditions: 1-4 mg/ml antibody in 50 mM sodium phosphate, pH 7.5, 10-20 U/μl PNGase F (from New England Biolabs, using their unit definition), 24 48 h at 37° C. After deglycosylation, antibodies were buffer-exchanged (using ultrafiltration or dialysis) into 20 mM sodium acetate, pH 4.5. Conditions for pepsinolysis were as follows: 30% (by volume) pepsin agarose (settled bed volume, beads washed in 20 mM NaOAc, pH 4.5), 0.5-2 mg/ml IgG, 20 mM NaOAc, 260 mM KC1, 0.1% Triton X-100, pH 4.5. Reactions were incubated at 37° C. with agitation for an amount of time that had previously been optimized (MAB9647, 12 h; MAB208, 4.5 h; MAB602, 3.5 h). After pepsin-treatment, the fragments were recovered from the pepsin agarose by washing the resin with 0.1M NaOAc, pH 4.5. The products of the pepsin-cleavage were then concentrated and exchanged into 0.1 M Na₂HPO₄, 5 mM EDTA, pH 6.0, and then treated with 20 mM 2-mercaptoethylamine (MEA) in the same buffer for 90 min at 37° C. The MEA was then removed by dialyzing for 6 hours at 4° C. against 0.1 M Na₂PO₄, 5 mM EDTA, using a 10 kD cutoff membrane, and then residual MEA was removed by running sample over a desalting column (PD-1 0, Amersham-Pharmacia, Piscataway, N.J.). immediately after this step, the reduced F(ab)′ was treated with 20 mM N-ethylmaleimide or maleimide-activated biotin (Pierce product number 21901) for 2 h at room temperature, and the unincorprated NEM or biotin-maleimide was then removed by dialysis. The samples were concentrated and the Fab′ fragments were purified from other fragments by FPLC using a Superdex-75 gel filtration column (Amersham-Pharmacia). In the case of the NEM-treated Fab′ fragments, random biotinylation was as described below. Samples of the FPLC-purified Fab′ fragments were diluted 1:1 with non-reducing protein loading buffer (62.5 mM TrisHCl, 25% glycerol, 2% SDS, 0.01% Bromophenol Blue) and loaded onto a 4-20% gradient SDS polyacrylamide gel (Product number 161-1123, Biorad, Hercules, Calif.). SDS-PAGE was performed according to Laemmli (1970). In each case the ˜50 kD band corresponding to the Fab′ was observed, and the only observable contaminants correspond to the sizes of the light and cleaved heavy chains. Because these contaminants co-migrated with the Fab′ in a high resolution gel filtration column, they presumably correspond to a non-covalent but otherwise structurally native complex between the light and cleaved heavy chains, in which the disulfide bond that normally links them has been reduced and alkylated. These fragments are likely to be functional since the disulfide bond between the heavy and light chain is not in the antigen-binding site. They correspond to about 20% of the purified protein, and some of these Fab′ fragments are >80% active.

The biotinylation of all capture agents used in this study was verified by Western blot analysis using an HRP-conjugated streptavidin (SA) probe (data not shown). In addition, the extent of biotinylation was estimated by using a SA resin pull-down assay (data not shown). Biotinylation was 60% or greater in each of these reactions. No attempt was made to remove non biotinylated protein prior to surface immobilization.

### Example 9

This example describes the carbobiotinylation protocol. IgG (3-5 mg/ml) were dialyzed into coupling buffer (0.1 M NaOAc pH 5.5) and then incubated with 20 mM sodium meta periodate, NaIO₄ in the dark for 1 h at 0° C. to oxidize the vicinal diols in the carbohydrate to aldehydes. The reaction was then quenched by the addition of 30 mM glycerol for 10 minutes, filtered to remove insoluble salts, and then dialyzed against coupling buffer for 6 hours. The aldehyde reactive probe (ARP, N-(aminooxyacetyl)-N′-(D biotinyl) hydrazine, trifluoroacetic acid salt, Molecular Probes, Eugene, Oreg.) is added at 1 mg/ml and incubated at room temperature for 2 h, after which the sample is extensively dialyzed against PBS.

### Example 10

This example describes the random biotinylation of IgG and Fab ′fragment molecules. IgG and NEM-treated Fab′ fragments were modified with the amine reactive probe, EZ Link™ Sulfo-NHS-Biotin (Pierce). Reactions were performed with a 20-fold molar excess of biotinylation reagent over protein in 1×PBS at room temperature for 2 hours. The biotinylation reagent was then quenched by adding Tris, pH 7.4, to a final concentration of 10 mM. The samples were then dialyzed against a 1000-fold excess of PBS 5 times in order to remove free biotin probe. After dialysis, the biotinylated proteins were analyzed on a gel and tested for extent of biotinylation on UltraLink™ Plus Immobilized Streptavidin Gel (Pierce). Typically 60% -100% of the protein would be biotinylated (data not shown).

### Example 11

This example describes using surface plasmon resonance (SPR) to measure surface coverage and activity of affinity agents linked to a thinfilm formed on a gold substrate. All SPR assays were performed in a BIAcore 3000 sing a biotinylated self-assembled monolayer formed on a gold-coated glass surface. The surface was prepared using unsymmetrical alkanedisulfide compound 10 according to the protocol of example 1. The prepared surface comprised N-hydroxysuccinimide and methoxy groups on its exposed face. This monolayer was then reacted with (+)-biotinyl-3,6-dioxaoctanediamine (tri-ethyleneglycol amino biotin) reagent to give a biotinylated surface. The biotin groups on the surface allow for the binding of streptavidin (SA). All assays were performed at 25° C. in PBS with 0.05% Tween-20. SA was loaded onto the surface at a flow rate of 20 μl/min at 0.1 mg/ml. Typically 320 μl was loaded to achieve a saturated surface of SA whereby a surface coverage of 3.7-4.0 pmol/cm² was obtained, as calculated according to Jung et al., Langmuir 14:5636-5648 (1998). After SA deposition, the various capture agents were loaded at 20-100 nM at a flow rate of 20 μl/min until saturation was observed. For analyte binding, flow rates of 80 μl/min were used unless otherwise noted. Various analyte concentrations over a broad range were assayed in order to determine the upper limit of analyte binding. For comparison, non-specific binding of the analytes to SA was tested independently at all analyte concentrations studied.

### Example 12

This example describes the synthesis of asymmetric NHS disulfide 120. With reference to FIG. 7, alkenyl bromide compound 100 is used as the starting material for both “arms” of the asymmetric disulfide. To produce the protein resistant arm, 100 is reacted with alkoxide triethylene glycol monomethyl ether (produced by deprotonation with NaH) to afford compound 102 in 60% yield. Compound 102 is subjected to radical addition to give the terminal thioacetyl compound 104 in 88% yield. The acetyl group is removed under acidic conditions (4N HCl/dioxane at 75° C. for 4 hours) to afford thiol 106 (95% ), which is treated with 2′,2′-dipyridyl disulfide to furnish the corresponding 2′-pyridyl mixed disulfide 108 in 98% yield.

The NHS “arm” of the mixed disulfide 120 is prepared from 100 via reflux with tetraethylene glycol in aqueous NaOH for 24 hours to yield compound 110 in 42% yield. Terminal alcohol 110 is treated with NaH and alkylated with t-butylbromoacetate to afford terminal ester compound 112 in 42% yield. Compound 112 is converted to thioacetyl compound 114 in 87% yield via free radical addition of thioacetic acid. Removal of the acetyl and t-butyl blocking groups by treatment with 2N aqueous HCl for 48 to 60 hours at 75° C. produced thiol 116 in 80% yield.

Compounds 108 and 116 were then combined in the presence of Amberlite CG-50 (pH ca. 3-4) for about 12 to about 16 hours at room temperature to yield asymmetric disulfide 118, which was converted to desired compound 120 via DCC-catalyzed condensation with N-hydroxysuccinimide.

1. A method for functionalizing a substrate, comprising:
providing a substrate;
contacting the substrate with a first reagent having at least first and second reactive functional groups such that the first reagent is operationally associated with the substrate by the first functional group thereby forming a monolayer;
associating a second reagent with the monolayer, the second reagent comprising a first functional group for association with the monolayer and at least a second functional group; and
associating a protein resistant reagent with the monolayer.

2. The method according to claim 1, wherein the first reagent is covalently bound to the substrate.

3. The method according to claim 1, wherein the first reagent is associated with the substrate by means of a functional group selected from the group consisting of a sulfur-containing group, siloxane, phosphine, phosphoric acid; phosphonic acid; hydroxamic acid, carboxylic acid, and combinations thereof.

4. The method according to claim 1, wherein the first reagent comprises one or more of a functional group selected from the group consisting of a sulfur-containing group, ester, activated ester, Michael acceptor, siloxane, silyl halide, phosphine, amine, azide, α, β-unsaturated ketone, hydroxyl, sulfhydryl, thiosemicarbazide, hydrazide, aminooxy group, aldehyde, alkyl halide, hydroxamic acid, and carboxylic acid.

5. The method according to claim 1, wherein the second reagent is covalently attached to the monolayer by means of an alkylation, chemoselective ligation, Michael addition, organometallic reaction, cycloaddition or acylation reaction.

6. The method according to claim 1, wherein the second reagent comprises one or more of a phosphine, amine, azide, α, β-unsaturated ketone, hydroxyl, sulfhydryl, thiosemicarbazide, hydrazide, aminooxy group, aldehyde, or alkyl halide.

7. The method according to claim 1, wherein the protein resistant component is covalently attached to the monolayer by means of an alkylation, Michael, Staudinger ligation, organometallic, cycloaddition, or acylation reaction.

8. The method according to claim 1, wherein the substrate is selected from the group consisting of gold, platinum, silver, copper, glass, silicon, silicon oxide, silicon nitride, tantalum oxide, titanium oxide, indium tin oxide, magnesium oxide, alumina, quartz, silica and combinations thereof.

9. The method according to claim 1, wherein the substrate comprises a noble metal and the first functional group of the first reagent is a sulfur-containing group.

10. The method according to claim 9, wherein the sulfur-containing group comprises a sulfhydryl group or a disulfide group.

11. The method according to claim 1, wherein the substrate comprises glass or silicon and the first functional group of the first reagent comprises a siloxane or silyl halide group.

12. The method according to claim 1, wherein the second reactive functional group of the first reagent includes one or more azide, amine, maleimide, phosphine, amine, azide, α, β-unsaturated ketone, hydroxyl, sulfhydryl, thiosemicarbazide, hydrazide, aminooxy group, aldehyde, alkyl halide, carboxy or activated ester groups.

13. The method according to claim 1, wherein the protein resistant reagent comprises at least one of a polyalkylene oxide group, polysulfone, polysaccharide, or a phosphocholine group.

14. The method according to claim 1, wherein the protein resistant reagent comprises a polyethylene glycol group.

15. The method according to claim 1, further comprising associating an affinity agent to the second functional group of the second reagent.

16. The method according to claim 15, further comprising quenching any remaining second functional groups.

17. The method according to claim 15, wherein the affinity agent comprises a protein.

18. The method according to claim 15, wherein associating the affinity agent to the second functional group of the second reagent comprises forming a covalent bond.

19. The method according to claim 18, wherein the covalent bond is formed by a reaction selected from the group consisting of alkylation, chemoselective ligation, Michael addition, organometallic reaction, cycloaddition and acylation reactions.

20. The method according to claim 18, wherein the covalent bond is formed by a Staudinger ligation reaction.

21. The method according to claim 15, wherein the second functional group of the second reagent comprises a biotin group and the affinity agent is a streptavidin.

22. The method according to claim 21, further comprising associating a biotinylated affinity agent to the streptavidin.

23. The method according to claim 22, wherein the biotinylated affinity agent is a biotinylated antibody.

24. A method for forming a monolayer, comprising:
providing a substrate;
contacting the substrate with an asymmetric disulfide, having a reactive functional group and a protein resistant functional group, thereby forming a monolayer on the substrate.

25. The method according to claim 24, wherein the asymmetric disulfide has the formula

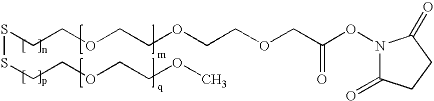

26. The method according to claim 24, wherein the asymmetric disulfide has the formula

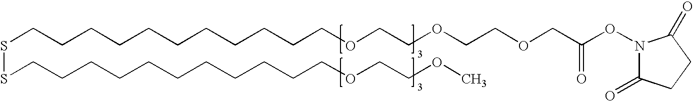

27. The method according to claim 24, further comprising contacting the substrate with a second disulfide thereby incorporating two corresponding sulfides into the monolayer.

28. An organic thinfilm array, comprising:
a substrate;
plural first reagents having first and second ends, the first end of the first reagents being operatively associated with the substrate;
plural second reagents, each second reagent having first and second ends and the first ends of the second reagent being operatively associated with the second end of the first reagent; and
a third reagent having a first end operatively associated with the second end of the first reagent and a second end, the third reagent comprising a protein resistant functional group.

29. The array according to claim 28, wherein the substrate is selected from the group consisting of gold, platinum, silver, copper, glass, silicon, silicon oxide, silicon nitride, tantalum oxide, titanium oxide, indium tin oxide, magnesium oxide, alumina, quartz, silica and combinations thereof.

30. The array according to claim 28, wherein the first reagent is covalently bound to the substrate.

31. The array according to claim 28, wherein the first reagent is associated with the substrate by means of a sulfur-containing group, siloxane, phosphine, hydroxamic acid, carboxylic acid, and combinations thereof.

32. The array according to claim 28, wherein the first reagent is associated with the substrate by at least one interaction comprising at least one of a metal-ligand bond, siloxane bond or a Coulombic interaction.

33. The array according to claim 28, wherein the wherein the second reagent is covalently attached to the first reagent.

34. The array according to claim 33, wherein the second reagent is covalently attached to the first reagent by means of an alkylation, chemoselective ligation, Michael addition, organometallic reaction, cycloaddition or acylation reaction.

35. The array according to claim 33, wherein the second reagent is covalently attached to the first reagent by means of a Staudinger ligation.

36. The array according to claim 33, wherein the second reagent is covalently attached to the first reagent by an amide, ether, ester, imide, sulfide, or carbon-carbon bond.

37. An array device comprising:
a substrate having an array of discrete array regions formed on the surface;
plural first reagents having first and second ends, the first end of the first reagents being operatively associated with the substrate;
plural second reagents, each second reagent having first and second ends and the first ends of the second reagent being operatively associated with the second end of the first reagent.
